(19) United States
(12) Patent Application Publication
(10) Pub. No.: US 2026/0176567 A1
(43) Pub. Date: Jun. 25, 2026

(54) BATCH CULTURE DEVICE

(71) Applicant: Panasonic Intellectual Property Management Co., Ltd., Osaka (JP)

(72) Inventors: SHOKO KUSAMA, Nara (JP); SEIJI KOJIMA, Nara (JP)

(21) Appl. No.: 19/543,911

(22) Filed: Feb. 19, 2026

Related U.S. Application Data
(63) Continuation of application No. PCT/JP2024/028028, filed on Aug. 6, 2024.

(30) Foreign Application Priority Data
Sep. 5, 2023 (JP) 2023-143877

(51) Int. Cl. C12M 1/00; C12M 3/00

(52) U.S. Cl. CPC C12M 31/10; C12M 21/02; C12M 23/02; C12M 23/46; C12M 41/14

(57) ABSTRACT

A batch culture device includes a reaction vessel capable of holding a culture solution of a photosynthetic microorganism, a shaft extending in a central portion of an inside of the reaction vessel along a side surface of the inside of the reaction vessel, and light sources disposed in the inside of the reaction vessel and including a first light source and a second light source. An angle θ1 between a first straight line and a second straight line in a plane perpendicular to a z direction in which the shaft extends satisfies a condition 90°<θ1≤180°, where the first straight line connects a center of the first light source and a center of the shaft when seen from the z direction, and the second straight line connects a center of the second light source and the center of the shaft when seen from the z direction.

### BACKGROUND

### 1. Technical Field

The present disclosure relates to a batch culture device.

### 2. Description of the Related Art

To date, culture devices for culturing photosynthetic microorganisms are known.

For example, International Publication No. 2014/132348 describes an organism culturing system for culturing an organism that performs photosynthesis, such as a microalga. The organism culturing system includes a liquid storage vessel, a culture vessel, a first light amount measuring unit, and a controller. The liquid storage vessel stores a light-absorbing solution that absorbs more light in a second wavelength band, whose wavelength is shorter than that of a first wavelength band, than light in the first wavelength band. The culture vessel is set in the inside of the liquid storage vessel and stores a culture solution, including a photosynthetic organism to be cultured, in the inside thereof. The first light amount measuring unit measures the amount of light that the culture vessel receives. Based on the measurement result of the first light amount measuring unit, the controller controls the liquid depth from the liquid surface of the light-absorbing solution to the culture vessel in the inside of the liquid storage vessel.

Japanese Unexamined Patent Application Publication (Translation of PCT Application) No. 2022-517443 describes a scalable vertical unit for culturing a photosynthetic microorganism. The vertical unit includes at least one sealable photobioreactor, a column operably engaged with the photobioreactor, and at least one light source. The at least one light source and the column are aligned along a longitudinal axis of the photobioreactor. The column is configured to control parameters including the temperature of the inside of the photobioreactor, the intensity of light emitted from the light source, the duration of illumination by the light source, the frequency of illumination, and the wavelength of light emitted by the light source.

Japanese Unexamined Patent Application Publication No. 2012-23990 describes a photobioreactor that is used in a circulating culture method of culturing photosynthetic microalgae. The photobioreactor includes a first culture unit, a second culture unit, and a pump unit. The pump unit is coupled to the first culture unit and the second culture unit so as to circulate a culture solution between the first culture unit and the second culture unit. In the first culture unit, a culture tank and a first light source are joined. The first light source emits light that allows photosynthetic microalgae to perform photosynthesis during culturing. The first light source is disposed in the inside of the culture tank. The first light source may be disposed on an outer surface of the culture tank.

### SUMMARY

One non-limiting and exemplary embodiment provides a batch culture device that is advantageous in terms of efficiency and scale-up of culturing of a photosynthetic microorganism.

In one general aspect, the techniques disclosed here feature a batch culture device including a reaction vessel that is capable of holding a culture solution of a photosynthetic microorganism, a shaft that extends in a central portion of an inside of the reaction vessel along a side surface of the inside of the reaction vessel, and light sources that are disposed in the inside of the reaction vessel. The light sources include a first light source and a second light source. An angle θ1 between a first straight line and a second straight line in a plane perpendicular to a z direction satisfies a condition 90°<θ1≤180°, where the z direction is a direction in which the shaft extends, the first straight line is a straight line that connects a center of the first light source and a center of the shaft when seen from the z direction, and the second straight line is a straight line that connects a center of the second light source and the center of the shaft when seen from the z direction.

With the present disclosure, it is possible to provide a batch culture device that is advantageous in terms of efficiency and scale-up of culturing of a photosynthetic microorganism.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 schematically illustrates an example of a batch culture device according to the present disclosure;

FIG. 2 schematically illustrates an example of the arrangement of light sources in the batch culture device according to the present disclosure;

FIG. 3 schematically illustrates another example of the arrangement of light sources in the batch culture device according to the present disclosure;

FIG. 4 schematically illustrates still another example of the arrangement of light sources in the batch culture device according to the present disclosure;

FIG. 5 schematically illustrates still another example of the arrangement of light sources in the batch culture device according to the present disclosure;

FIG. 6 schematically illustrates still another example of the arrangement of light sources in the batch culture device according to the present disclosure;

FIG. 7 schematically illustrates an example of the arrangement of a light source in a batch culture device according to a comparative example;

FIG. 8 schematically illustrates a batch culture device according to another comparative example;

FIG. 9 is a plan view illustrating a bracket of a batch culture device according to the present disclosure;

FIG. 10 schematically illustrates another example of a batch culture device according to the present disclosure;

FIG. 11 schematically illustrates still another example of a batch culture device according to the present disclosure; and

FIG. 12 is a graph illustrating the relationship between the specific growth rate of a photosynthetic microorganism and the number of light sources.

### DETAILED DESCRIPTIONS

### Underlying Knowledge Forming Basis of the Present Disclosure

As social demand for decarbonization has been increasing, substance production using a photosynthetic microorganism is attracting attention as a next-generation technology with low-environmental load. Photosynthesis is a reaction that produces an organic substance from water and carbon dioxide in air by using light energy at room temperature and pressure. Combined with development of genetic engineering technology in recent years, it has been reported that productivity in producing various chemical compounds has improved by manipulating genes of a photosynthetic microorganism that are related to metabolism or substance biosynthesis.

It is considered that the light amount of a light source is important in culturing a photosynthetic microorganism. For example, in the organism culturing system described in International Publication No. 2014/132348, the amount of light that the culture vessel receives is measured, and the liquid depth from the liquid surface of the light-absorbing solution to the culture vessel is controlled. In the vertical unit described in Japanese Unexamined Patent Application Publication (Translation of PCT Application) No. 2022-517443, parameters related to light emitted by the light source are controlled. The photobioreactor described in Japanese Unexamined Patent Application Publication No. 2012-23990 is used in a circulating culture method of culturing photosynthetic microalgae, and it is not supposed that the photobioreactor be used for culturing a photosynthetic microorganism by a batch operation.

According to a study carried out by the inventors, it has been confirmed that, when a photosynthetic microorganism is to be cultured by a batch operation to achieve scale-up suitable for commercial use, it is difficult to culture the photosynthetic microorganism efficiently only by optimizing the light amount of a light source.

The inventors have investigated to develop a batch culture device whose efficiency of culturing a photosynthetic microorganism can be easily increased while achieving scale-up. As a result, the inventors have found that the efficiency of culturing a photosynthetic microorganism can be easily increased while achieving scale-up by disposing light sources under a predetermined condition in the inside of a reaction vessel of a batch culture device. Base on this novel finding, the idea of a batch culture device according to the present disclosure has been obtained.

### Embodiments

Hereafter, embodiments of the present disclosure will be described with reference to the drawings. The present disclosure is not limited to the following embodiments. In the drawings, the z direction represents the gravitational direction.

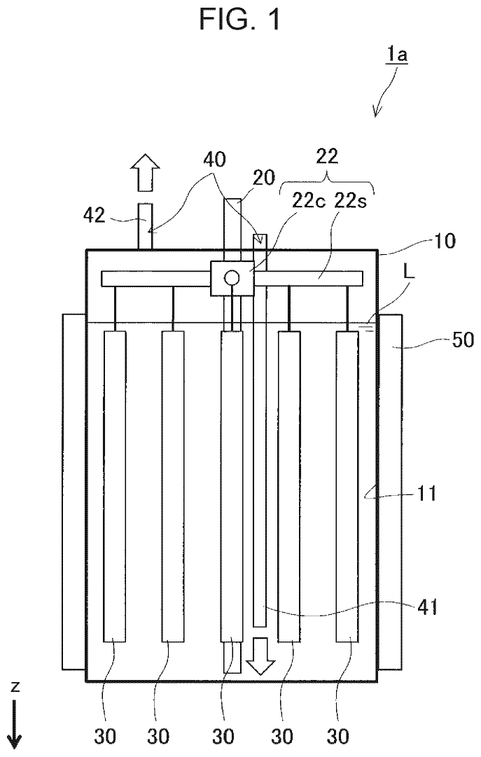

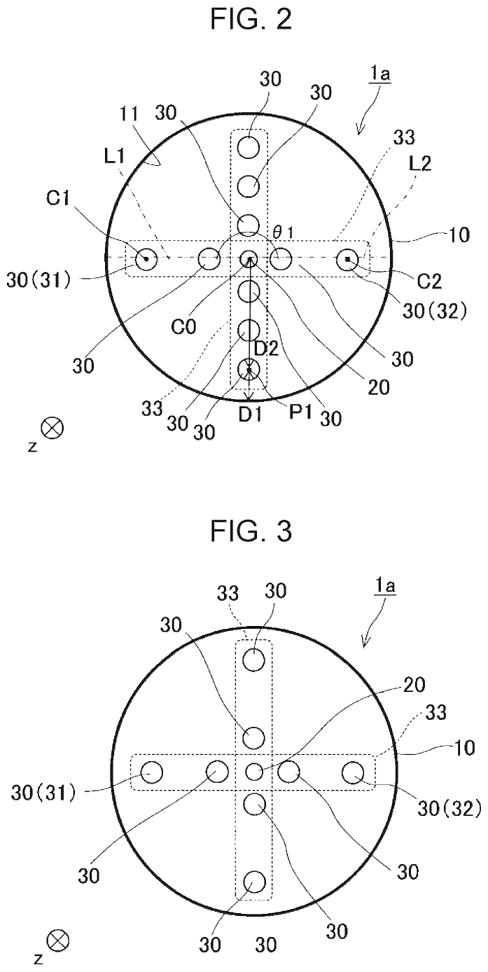

FIG. 1 schematically illustrates an example of a batch culture device according to the present disclosure. As illustrated in FIG. 1, a batch culture device 1a includes a reaction vessel 10, a shaft 20, and light sources 30. The reaction vessel 10 is capable of holding a culture solution of a photosynthetic microorganism. The culture solution incudes, for example, a photosynthetic microorganism and a culture medium. The shaft 20 extends in a central portion in the reaction vessel 10 along a side surface 11 of the inside of the reaction vessel 10. The light sources 30 are disposed in the inside of the reaction vessel 10. FIG. 2 schematically illustrates an example of the arrangement of the light sources 30 in the batch culture device 1a. This figure illustrates the shaft 20 and the light sources 30 as seen from the direction in which the shaft 20 extends. As illustrated in FIG. 2, the light sources 30 include a first light source 31 and a second light source 32. In FIG. 2, the angle θ1 between a first straight line L1 and a second straight line L2 in a plane perpendicular to the z direction satisfies the condition 90°<θ1≤180°. The first straight line L1 is a straight line that connects the center C1 of the first light source 31 and the center C0 of the shaft 20 when seen from the z direction. The second straight line L2 is a straight line that connects the center C2 of the second light source 32 and the center C0 of the shaft 20 when seen from the z direction. Each of the light sources 30 may be shaped like a cylinder. The center of each of the light sources 30 may include the center of the bottom surface of the cylinder and/or the center of the top surface of the cylinder, and may be included in a straight line that is parallel to the Z axis. The angle θ1 may be the angle between a plane including the center C1 and the shaft 20 and a plane including the center C2 and the shaft 20.

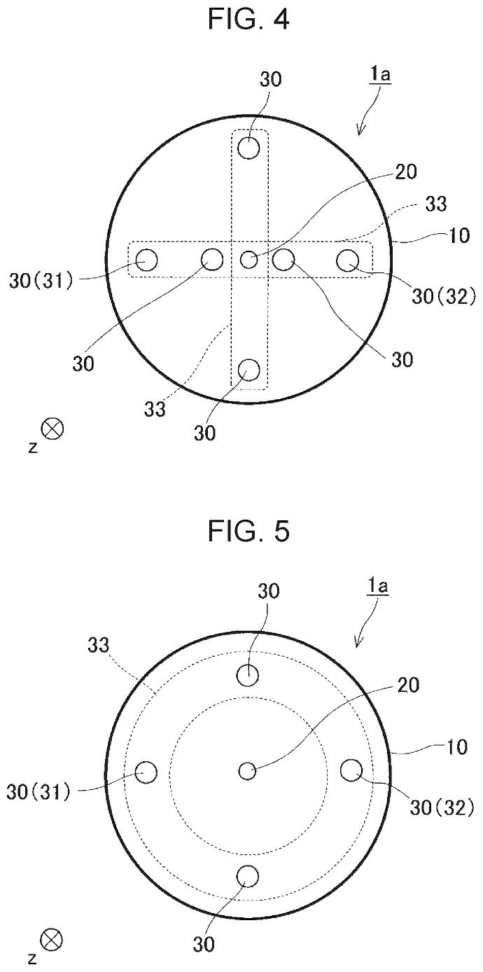

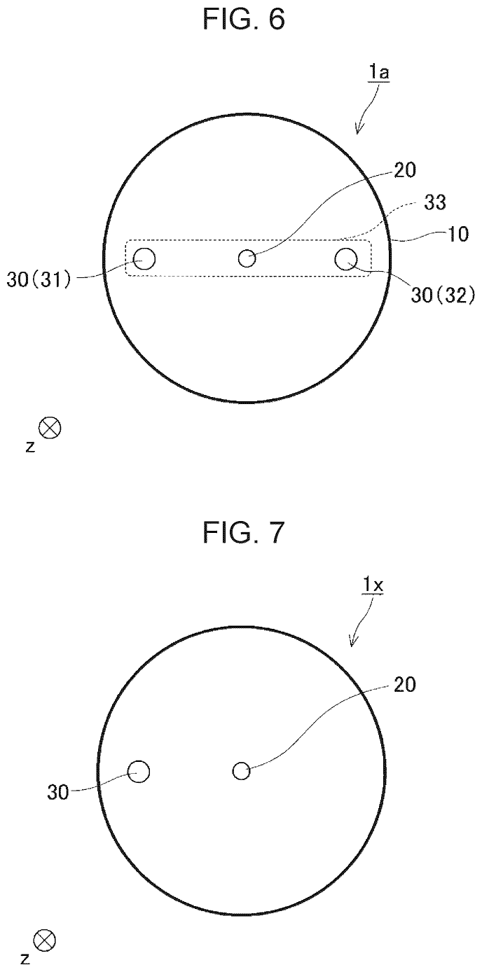

The number and the arrangement of the light sources 30 are not particularly limited, as long as the light sources 30 include the first light source 31 and the second light source 32 that satisfy the condition 90°<θ1≤180°. As illustrated in FIG. 2, the number of the light sources 30 is, for example, ten. Each of FIGS. 3, 4, 5, and 6 illustrates another example the arrangement of the light sources 30 in the batch culture device 1a. As illustrated in FIG. 3, the number of the light sources 30 may be eight. As illustrated in FIG. 4, the number of the light sources 30 may be six. As illustrated in FIG. 5, the number of the light sources 30 may be four. As illustrated in FIG. 6, the number of the light sources 30 may be two. The number of the light sources 30 may be three, five, seven, nine, or greater than or equal to eleven. The number of the light sources 30 is, for example, less than or equal to forty-eight.

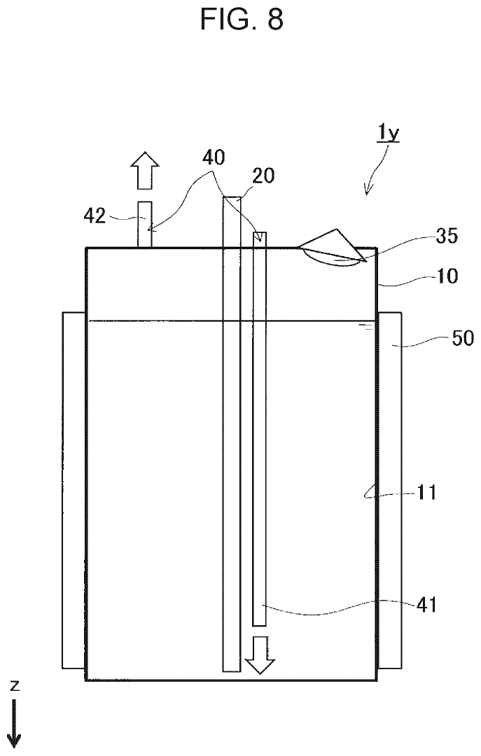

FIG. 7 schematically illustrates an example of the arrangement of a light source in a batch culture device 1x according to a comparative example. The batch culture device 1x is configured in the same way as the batch culture device 1a unless otherwise noted. The batch culture device 1x includes, as a light source, only one light source 30. FIG. 8 schematically illustrates a batch culture device 1y according to another comparative example. The batch culture device 1y is configured in the same way as the batch culture device 1a unless otherwise noted. The batch culture device 1y includes, as a light source, only a light source 35 that is disposed in an upper portion of the reaction vessel 10.

In the batch culture devices 1x and 1y, the number of the light source is one. When these batch culture devices have scales suitable for commercial use, even if the light amount of the light source 30 or 35 is adjusted, it is difficult for a desired amount of light from the light source 30 or 35 to reach a large region in the culture solution held in the reaction vessel 10. On the other hand, in the batch culture device 1a, the light sources 30 include the first light source 31 and the second light source 32 that satisfy the condition 90°<θ1≤180°. Therefore, even when the batch culture device 1a has a scale suitable for commercial use, a desired amount of light from the light sources 30 can easily reach a large region in the culture solution of a photosynthetic microorganism held in the reaction vessel 10. Therefore, the efficiency of culturing a photosynthetic microorganism tends to increase.

The photosynthetic microorganism is not limited to a specific photosynthetic microorganism. The photosynthetic microorganism is, for example, a cyanobacterium. Cyanobacteria, which are also called blue-green algae or blue-green bacteria, are a group of procaryotes that perform photosynthesis while generating oxygen by decomposing water by using energy obtained by collecting photoenergy with chlorophyll. Cyanobacteria are diverse and include, for example, in terms of cell shape, a unicellular species such as Synechocystis sp. PCC 6803 and a filamentous species in which multiple cells are connected such as Anabaena sp. PCC 7120. Also in terms of growing environment, cyanobacteria include a thermophilic species such as Thermosynechococcus elongatus, a marine species such as Synechococcus elongatus, and a freshwater species such as Synechocystis. Cyanobacteria further include many species having unique features, such as: a species that has a gas vesicle and generates a toxin, such as Microcystis aeruginosa; and a species that has a protein complex called phycobilisome, which is a light-harvesting antenna, in a protoplasmic membrane, such as Gloeobacter violaceus.

The angle θ1 preferably satisfies the condition 105°≤θ1≤180°, more preferably satisfies the condition 120°≤θ1≤180°, and further preferably satisfies the condition 135°≤θ1≤180°. The angle θ1 particularly preferably satisfies the condition 150°≤θ1≤180°, and further particularly preferably satisfies the condition 165°≤θ1≤180°.

As illustrated in FIGS. 2 to 6, the light sources 30 include, for example, a light source group 33. The light source group 33 includes light sources 30 that are arranged rotationally symmetrically around the shaft 20 when the shaft 20 and the light sources 30 are seen along the shaft 20. With such a configuration, a desired amount of light from the light sources 30 can more easily reach a large region in the culture solution of a photosynthetic microorganism held in the reaction vessel 10. Therefore, the efficiency of culturing a photosynthetic microorganism tends to increase further.

The light sources 30 include, for example, two light source groups 33. Each light source group 33 includes light sources 30 arranged at positions that are two-fold or four-fold rotationally symmetric around the shaft 20 when the shaft 20 and the light sources 30 are seen along the shaft 20.

The reaction vessel 10 is not limited to a specific vessel as long as the reaction vessel 10 is capable of holding a culture solution of a photosynthetic microorganism. The reaction vessel 10 has, for example, pressure resistance. When a photosynthetic microorganism is to be cultured by a batch operation, the inside of the reaction vessel 10 needs to be sterilized before the photosynthetic microorganism is cultured. This sterilization can be performed in a state in which the pressure in the reaction vessel 10 is higher than the atmospheric pressure. Therefore, because the reaction vessel 10 has pressure resistance, the inside of the reaction vessel 10 can be easily sterilized, and the efficiency of culturing a photosynthetic microorganism tends to increase. The reaction vessel 10 can be used, for example, with a pressure higher than or equal to 0.2 MPa.

The inner surface of the reaction vessel 10 is made of, for example, a metal. The inner surface of the reaction vessel 10 may be a mirror surface. In this case, light emitted from the light sources 30 is easily and efficiently used for culturing a photosynthetic microorganism, and the efficiency of culturing a photosynthetic microorganism tends to increase further.

The side surface 11 of the inside of the reaction vessel 10 is, for example, a columnar surface. The side surface 11 of the inside of the reaction vessel 10 may be a cylindrical surface. The position of the shaft 20 in the reaction vessel 10 may be at the center of the inside of the reaction vessel 10 in a plane perpendicular to the shaft 20 or may be displaced from the center. For example, when the side surface 11 of the inside of the reaction vessel 10 is a cylindrical surface, the distance between the center of the inside of the reaction vessel 10 and the center C0 of the shaft 20 is less than or equal to 10% of the inside diameter of the reaction vessel 10.

The reaction vessel 10 is capable of holding, for example, at least 0.4 m³ of a culture solution. Even though the reaction vessel 10 has a scale suitable for commercial use in this way, because the light sources 30 include the first light source 31 and the second light source 32 that satisfy the condition 90°<θ1≤180° as described above, the efficiency of culturing a photosynthetic microorganism tends to increase. The capacity of the reaction vessel 10 is, for example, greater than or equal to 0.45 m³, and may be greater than or equal 0.5 m³, greater than or equal to 1.0 m³, or greater than or equal to 2.0 m³. The capacity of the reaction vessel 10 is, for example, less than or equal to 5 m³.

As illustrated in FIG. 1, the shaft 20 extends, for example, from the outside of the reaction vessel 10 to the vicinity of the bottom surface of the reaction vessel 10 through the upper wall of the reaction vessel 10. The shaft 20 is made of, for example, a metal. The shaft 20 does not move while a photosynthetic microorganism is cultured. For example, a mixing impeller (not shown) can be attached to the shaft 20. In addition, the shaft 20 can be coupled to a motor (not shown) outside of the reaction vessel 10. For example, when the inside of the reaction vessel 10 is to be cleaned, the mixing impeller is attached to the shaft 20, and the shaft 20 is connected to the motor. Thus, it is possible to agitate a cleaning liquid when the inside of the reaction vessel 10 is to be cleaned, and the reaction vessel 10 can be efficiently cleaned.

As illustrated in FIG. 2, the light sources 30 include, for example, a light source 30 that is disposed at a first position P1 when the shaft 20 and the light sources 30 are seen along the shaft 20. The distance D1 between the side surface 11 of the reaction vessel 10 and the first position P1 is shorter than the distance D2 between the center of the shaft 20 and the first position P1. With such a configuration, a desired amount of light from the light source 30 can easily reach the vicinity of the side surface 11 of the reaction vessel 10, and the efficiency of culturing a photosynthetic microorganism tends to increase further.

The first position P1 preferably satisfies the condition D1≤(D1+D2)/3. With such a configuration, a desired amount of light from the light source 30 can more easily reach the vicinity of the side surface 11 of the reaction vessel 10, and the efficiency of culturing a photosynthetic microorganism tends to increase further. The first position P1 satisfies, for example, the condition (D1+D2)/10≤D1.

For example, the first light source 31 is disposed at a position such that the distance between the side surface 11 of the reaction vessel 10 and the first light source 31 is shorter than the distance between the center of the shaft 20 and the first light source 31 when the shaft 20 and the light sources 30 are seen along the shaft 20. In addition, the second light source 32 is disposed at a position such that the distance between the side surface 11 of the reaction vessel 10 and the second light source 32 is shorter than the distance between the center of the shaft 20 and the second light source 32. With such a configuration, the efficiency of culturing a photosynthetic microorganism tends to increase further. The distance between the side surface 11 and the first light source 31 and the distance between the center of the shaft 20 and the first light source 31 may satisfy a condition similar to that satisfied by the distance D1 and the distance D2. The distance between the side surface 11 and the second light source 32 and the distance between the center of the shaft 20 and the second light source 32 may satisfy a condition similar to that satisfied by the distance D1 and the distance D2.

As illustrated in FIG. 1, for example, when the inside of the reaction vessel 10 is filled with a liquid whose volume is greater than or equal to 60% and less than or equal to 90% of the capacity of the reaction vessel 10, a liquid surface L is formed by the liquid. As illustrated in FIG. 1, the light sources 30 include, for example, a light source 30 that is disposed between the liquid surface L and the bottom surface of the reaction vessel 10. In culturing a photosynthetic microorganism, a culture solution whose volume is greater than or equal to 60% and less than or equal to 90% of the capacity of the reaction vessel 10 can be held in the inside of the reaction vessel 10. Therefore, because the light sources 30 include a light source 30 that is disposed between the liquid surface L and the bottom surface of the reaction vessel 10, a desired amount of light from the light sources 30 can easily reach a large region in the culture solution. As a result, the efficiency of culturing a photosynthetic microorganism tends to increase further.

For example, when the inside of the reaction vessel 10 is filled with a liquid whose volume is greater than or equal to 60% and less than or equal to 90% of the capacity of the reaction vessel 10 as described above, the area of the light-emitting surfaces the light sources 30 that are in contact with the liquid is greater than or equal to a half of the total area of the light-emitting surfaces. With such a configuration, a desired amount of light from the light sources 30 can easily reach a large region in the culture solution held in the inside of the reaction vessel 10. As a result, the efficiency of culturing a photosynthetic microorganism tends to increase further.

The shape of the light sources 30 is not limited to a specific shape. The light sources 30 extend, for example, along the shaft 20. With such a configuration, a desired amount of light from the light sources 30 can easily reach a large region in the culture solution held in the inside of the reaction vessel 10. As a result, the efficiency of culturing a photosynthetic microorganism tends to increase further.

The light-emitting surface of the light sources 30 is, for example, a columnar surface, and may be a cylindrical surface.

The wavelength λ of light emitted from the light sources 30 is not limited to a specific value. The wavelength λ may be, for example, greater than or equal to 400 nm and less than or equal to 700 nm. In this case, the efficiency of culturing a photosynthetic microorganism tends to increase further.

As illustrated in FIG. 1, the batch culture device 1a further includes, for example, a bracket 22. The bracket 22 is attached to the shaft 20. The light sources 30 are suspended from the bracket 22. With such a configuration, it is easy to dispose the light sources 30 at desired positions by using the bracket 22. Moreover, for example, when the inside of the reaction vessel 10 is to be cleaned and sterilized, it is easy to take the light sources 30 out of the reaction vessel 10 together with the bracket 22.

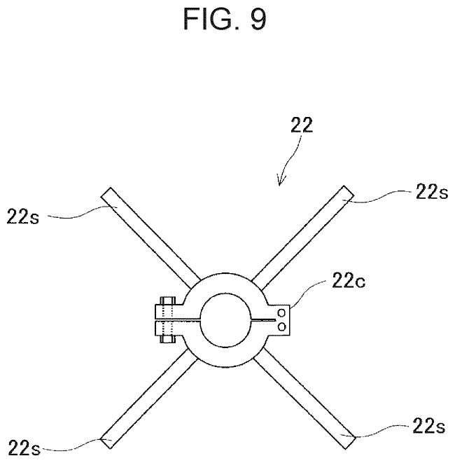

FIG. 9 is a plan view illustrating the bracket 22 of the batch culture device 1a. As illustrated in FIG. 9, the bracket 22 includes a clamp 22c, which is removable from the shaft 20, and a bar 22s. The bar 22s extends from the clamp 22c toward the side surface 11 of the reaction vessel 10 when the clamp 22c is fixed to the shaft 20. With such a configuration, it is easy to perform an operation of arranging the light sources 30 in the inside of the reaction vessel 10 and an operation of taking the light sources 30 out of the reaction vessel 10.

The bar 22s may be removable from the clamp 22c. In this case, for example, by only replacing the bar 22s, it is possible to change the number and the arrangement of the light sources 30 in the reaction vessel 10 in accordance with the reaction vessel 10. The bar 22s may be integrated with the clamp 22c.

As illustrated in FIG. 1, the batch culture device 1a further includes, for example, a temperature adjuster 50. The temperature adjuster 50 is disposed along the inner surface of the reaction vessel 10, and adjusts the temperature of the culture solution. With such a configuration, it is easy to adjust the temperature of the culture solution in the batch culture device 1a to a temperature suitable for culturing a photosynthetic microorganism.

The temperature adjuster 50 adjusts, for example, the absolute value of the difference between the optimal growing temperature for a photosynthetic microorganism and the temperature of the culture solution in the reaction vessel 10 to less than or equal to 5° C. Thus, in the batch culture device 1a, the efficiency of culturing a photosynthetic microorganism tends to increase further.

As illustrated in FIG. 1, the temperature adjuster 50 is disposed, for example, so as to surround the outer surface of the reaction vessel 10. With such a configuration, the temperature of the culture solution held in the reaction vessel 10 easily becomes uniform, and the efficiency of culturing a photosynthetic microorganism tends to increase further.

As described above, the light sources 30 extend, for example, along the shaft 20. The temperature adjuster 50 overlaps, for example, the light sources 30 in a direction parallel to the shaft 20. With such a configuration, the efficiency of culturing a photosynthetic microorganism tends to increase further.

The temperature adjuster 50 includes, for example, a water jacket. The temperature adjuster 50 may be an electric heater.

As illustrated in FIG. 1, the batch culture device 1a further includes, for example, an air passage 40. The air passage 40 guides air to a lower portion of the reaction vessel 10 and discharges air from an upper portion of the reaction vessel 10. With such a configuration, air is guided to a lower portion of the reaction vessel 10, and the efficiency of culturing a photosynthetic microorganism tends to increase further.

The batch culture device 1a includes, for example, a supply pipe 41 and a discharge pipe 42 that constitute the air passage 40. The supply pipe 41 extends from the outside of the reaction vessel 10 to the inside of the reaction vessel 10, and one end of the supply pipe 41 that is positioned in the inside of the reaction vessel 10 serves as a supply port. The discharge pipe 42 connects the inside of the reaction vessel 10 to the outside of the reaction vessel 10. The discharge pipe 42 has one end that is in contact with the inside of the reaction vessel 10, and the one end serves as a discharge port.

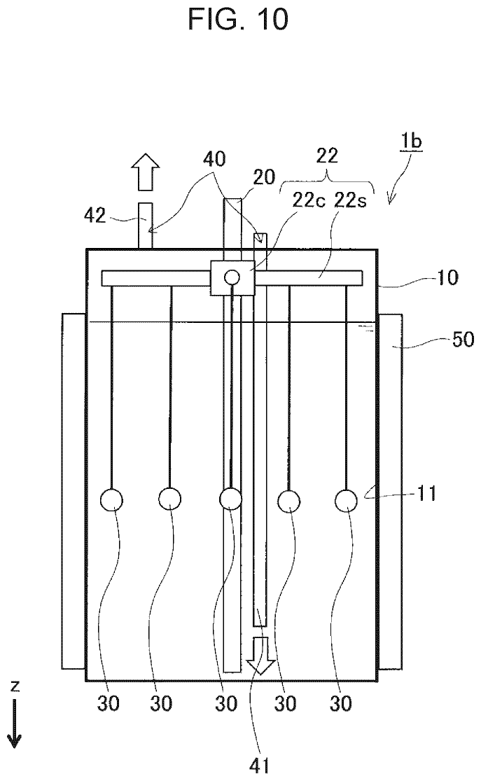

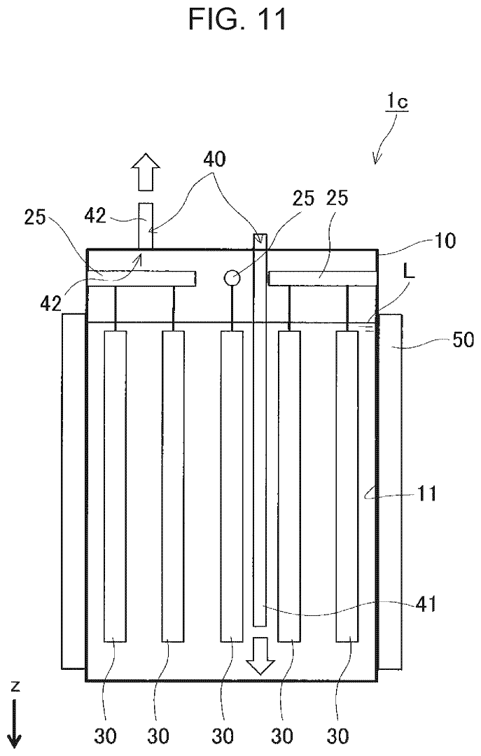

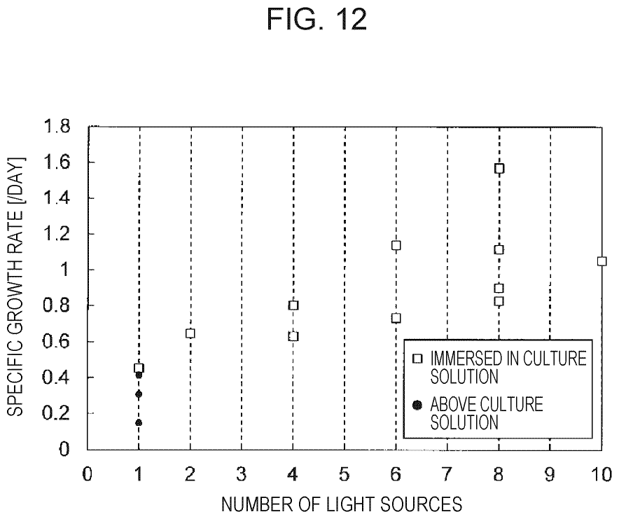

It is possible to modify the batch culture device 1a from various viewpoints. For example, the batch culture device 1a may be modified like a batch culture device 1b illustrated in FIG. 10 or a batch culture device 1c illustrated in FIG. 11. FIG. 10 schematically illustrates another example of a batch culture device according to the present disclosure, and FIG. 11 schematically illustrates still another example of a batch culture device according to the present disclosure. The batch culture devices 1b and 1c illustrated in FIGS. 10 and 11 are configured in the same way as the batch culture device 1a unless otherwise noted. Constituent elements of the batch culture devices 1b and 1c corresponding to those of the batch culture device 1a will be denoted by the same numerals, and detailed descriptions of such constituent elements will be omitted. Descriptions related to the batch culture device 1a also apply to the batch culture devices 1b and 1c unless technologically contradictory.

As illustrated in FIG. 10, in the batch culture device 1b, the light sources 30 are each spherical. Also in this case, because the light sources 30 include the first light source 31 and the second light source 32 that satisfy the condition 90°<θ1≤180°, the efficiency of culturing a photosynthetic microorganism tends to increase.

In the batch culture device 1b, the light sources 30, each of which is spherical, may be arranged in a direction parallel to the shaft 20.

As illustrated in FIG. 11, the batch culture device 1c includes, for example, a protruding member 25 that protrudes from the side surface 11 of the inside of the reaction vessel 10 toward the center of the inside of the reaction vessel 10. The light sources 30 are suspended from the protruding member 25. Thus, the light sources 30 can be disposed at desired positions. The batch culture device 1c does not include, for example, the shaft 20. The batch culture device 1c may include the shaft 20.

### APPENDIX

Based on the foregoing description, the following technologies are disclosed.

(Technology 1) A batch culture device comprising:
a reaction vessel that is capable of holding a culture solution of a photosynthetic microorganism;
a shaft that extends in a central portion of an inside of the reaction vessel along a side surface of the inside of the reaction vessel; and
light sources that are disposed in the inside of the reaction vessel,
wherein the light sources include a first light source and a second light source, and
wherein an angle θ1 between a first straight line and a second straight line in a plane perpendicular to a z direction satisfies a condition 90°<θ1≤180°, where the z direction is a direction in which the shaft extends, the first straight line is a straight line that connects a center of the first light source and a center of the shaft when seen from the z direction, and the second straight line is a straight line that connects a center of the second light source and the center of the shaft when seen from the z direction.

(Technology 2) The batch culture device according to Technology 1,
wherein the light sources include a light source group constituted by light sources that are disposed rotationally symmetrically around the shaft when the shaft and the light sources are seen along the shaft.

(Technology 3) The batch culture device according to Technology 1 or 2,
wherein the photosynthetic microorganism is a cyanobacterium.

(Technology 4) The batch culture device according to any one of Technologies 1 to 3,
wherein the reaction vessel has pressure resistance.

(Technology 5) The batch culture device according to any one of Technologies 1 to 4,
wherein the light sources include a light source that is disposed at a first position when the shaft and the light sources are seen along the shaft, and
wherein a distance between the side surface of the reaction vessel and the first position is shorter than a distance between the center of the shaft and the first position.

(Technology 6) The batch culture device according to any one of Technologies 1 to 5,
wherein the light sources include a light source that is disposed, when the inside of the reaction vessel is filled with a liquid whose volume is greater than or equal to 60% and less than or equal to 90% of a capacity of the reaction vessel, between a liquid surface formed by the liquid and a bottom surface of the reaction vessel.

(Technology 7) The batch culture device according to any one of Technologies 1 to 6,
wherein, when the inside of the reaction vessel is filled with a liquid whose volume is greater than or equal to 60% and less than or equal to 90% of a capacity of the reaction vessel, an area of light-emitting surfaces of the light sources that are in contact with the liquid is greater than or equal a half of a total area of the light-emitting surfaces.

(Technology 8) The batch culture device according to any one of Technologies 1 to 7,
wherein the light sources extend along the shaft.

(Technology 9) The batch culture device according to any one of Technologies 1 to 8, further comprising:
a bracket attached to the shaft,
wherein the light sources are suspended from the bracket.

(Technology 10) The batch culture device according to Technology 9,
wherein the bracket includes a clamp that is removable from the shaft and a bar that extends from the clamp toward the side surface of the reaction vessel when the clamp is fixed to the shaft.

(Technology 11) The batch culture device according to any one of Technologies 1 to 10, further comprising:
a temperature adjuster that is disposed along an inner surface of the reaction vessel and adjusts a temperature of the culture solution.

(Technology 12) The batch culture device according to Technology 11,
wherein the temperature adjuster adjusts an absolute value of a difference between an optimal growing temperature for the photosynthetic microorganism and the temperature of the culture solution to less than or equal to 5° C.

(Technology 13) The batch culture device according to Technology 11 or 12,
wherein the temperature adjuster is disposed so as surround an outer surface of the reaction vessel.

(Technology 14) The batch culture device according to any one of Technologies 11 to 13,
wherein the light sources extend along the shaft, and
wherein the temperature adjuster overlaps the light sources in a direction parallel to the shaft.

(Technology 15) The batch culture device according to any one of Technologies 11 to 14,
wherein the reaction vessel has an inner surface that is made of a metal or that is a mirror surface.

(Technology 16) The batch culture device according to any one of Technologies 1 to 15, further comprising:
an air passage that guides air to a lower portion of the reaction vessel and discharges air from an upper portion of the reaction vessel.

(Technology 17) The batch culture device according to any one of Technologies 1 to 16,
wherein the reaction vessel is capable of holding at least 0.4 m³ of the culture solution.

(Technology 18) The batch culture device according to any one of Technologies 1 to 17,
wherein the light sources emit light in a wavelength range of greater than or equal to 400 nm and less than or equal to 700 nm.

(Technology 19) A batch culture device comprising:
a reaction vessel that is capable of holding a culture solution of a photosynthetic microorganism;
light sources that are disposed in an inside of the reaction vessel; and
a protruding member that protrudes from a side surface of the inside of the reaction vessel toward a center of the inside of the reaction vessel,
wherein the light sources are suspended from the protruding member,
wherein the light sources include a first light source and a second light source, and
wherein an angle θ1 between a first straight line and a second straight line in a plane perpendicular to a z direction satisfies a condition 90°<θ1≤180°, where the z direction is a direction parallel to the side surface of the inside of the reaction vessel, the first straight line is a straight line that connects a center of the first light source and a center of the inside of the reaction vessel when seen from the z direction, and the second straight line is a straight line that connects a center of the second light source and the center of the inside of the reaction vessel when seen from the z direction.

### Examples

Hereafter, the present disclosure will be described in further detail by using Examples. The following Examples are described by way of illustration, and the present disclosure is not limited to the following Examples.

### EXAMPLES

A culture solution of Synechocystis was prepared by adding Synechocystis sp. PCC6803 (hereafter, referred to as “Synechocystis”), which is a species of cyanobacteria, to 0.4 m³ of BG-11 culture medium so that the initial bacterial cell concentration OD₇₃₀ became 0.02 to 0.05. The optimal growing temperature for Synechocystis is 30° C. The initial bacterial cell concentration OD₇₃₀ is the optical density of the culture solution at the wavelength of 730 nm. Table 1 shows the concentration of each component in BG-11 culture medium.

TABLE 1

| Component | Concentration [mg/L] |
|---|---|
| Ethylenediamine Tetraacetic Acid Disodium Salt Dihydrate | 1 |
| Ammonium Iron (III) Citrate, | 6 |
| Citric Acid | 6 |
| NaNO₃ | 1500 |
| MgSO₄ | 75 |
| K₂HPO₄ | 39 |
| CaCl₂ | 28.6 |
| H₃BO₃ | 2.86 |
| MnCl₂•4H₂O | 1.81 |
| ZnSO₄•7H₂O | 0.22 |
| CuSO₄•5H₂O | 0.08 |
| Na₂MoO₄•H₂O | 0.021 |
| Co (NO₃)₂•6H₂O | 0.0494 |
| HEPES (adjusted to pH 7.5 by using KOH) | 2383 |

The culture solution of Synechocystis described above was placed in the inside of a tank made of stainless steel SUS304 and having a capacity of 0.5 m³, and Synechocystis was cultured while maintaining the temperature of the inside of the tank at 30° C. The temperature of the inside of the tank was adjusted by using a water jacket disposed around the tank. A shaft made of stainless steel SUS304 was disposed in a central portion of the inside of the tank. The shaft extended to the vicinity of the bottom surface of the tank through the top surface of the tank. In addition, a bracket illustrated in FIG. 9 was attached to the shaft. As illustrated in FIGS. 2 to 6, ten, eight, six, four, or two light sources were suspended from a bar of the bracket and disposed rotationally symmetrically around the shaft in the inside of the tank. As each of the light source, an “LED Fishing Lamp Long White”, made by Ks garage Corporation, was used. This light source, whose power consumption was 60 W, was capable of emitting light in a wavelength range of greater than or equal to 400 nm and less than or equal to 700 nm. The light sources included a pair of light sources that were disposed at positions such that θ1=180° when the shaft and the light sources were seen along the shaft. The inside diameter of the tank was 0.73 m, and the distance between the side surface of the tank and a light source nearest to the side surface was 0.09 m. In culturing Synechocystis, the light sources were caused to emit light in a state in which the light sources were completely immersed in the culture solution. In culturing Synechocystis, air was drawn from a supply port positioned in the vicinity of the bottom surface of the tank, and air was discharged from a discharge port disposed in an upper portion of the tank. The flow rate of air supplied into the tank was adjusted to 0.4 m³/min.

### Comparative Example 1

As illustrated in FIG. 7, except that only one light source was disposed in the inside of the tank, Synechocystis was cultured in the same way as in Example.

### Comparative Example 2

Synechocystis was cultured in the same way as Example except for the following. As illustrated in FIG. 8, instead of the light sources described above, an LED floodlight “GINGA II” made by Atsuta Shizai Co., Ltd or an LED searchlight made by Art Pro LLC (power consumption 320 W) was disposed above the culture solution and caused to emit light. Only one light source was used in culturing Synechocystis.

### Specific Growth Rate

In the period of culturing Synechocystis, the optical density OD₇₃₀ of the culture solution at the wavelength of 730 nm was measured when culturing was started and when one day elapsed after culturing was started, and the specific growth rate μ [/day] of Synechocystis was determined in accordance with the following expression (1). In expression (1), OD_{730_0} is the optical density of the culture solution at the wavelength of 730 nm when culturing of Synechocystis was started, and OD_{730_1} is the optical density of the culture solution at the wavelength of 730 nm when one day elapsed after culturing of Synechocystis was started.

$$μ = ln ( O {D}_{730 \_ 1} - O {D}_{730 \_ 0} ) \qquad ( 1 )$$

FIG. 12 is a graph illustrating the relationship between the specific growth rate of Synechocystis, when Synechocystis was cultured as described above, and the number of light sources that were disposed in the inside of the tank. As illustrated in FIG. 12, when two or more light sources were disposed at predetermined positions and caused to emit light in a state of being completely immersed in the culture solution, the specific growth rate u was greater than or equal to 0.6 [/day], and the efficiency of culturing Synechocystis was high. On the other hand, when only one light source was used as in comparative example 1 and comparative example 2, the specific growth rate u was less than 0.6 [/day], and the efficiency of culturing Synechocystis was low.

With the present disclosure, it is possible to produce a substance by culturing a photosynthetic microorganism in a commercial scale.

What is claimed is:

1. A batch culture device comprising:
a reaction vessel that is capable of holding a culture solution of a photosynthetic microorganism;
a shaft that extends in a central portion of an inside of the reaction vessel along a side surface of the inside of the reaction vessel; and
light sources that are disposed in the inside of the reaction vessel,
wherein the light sources include a first light source and a second light source, and
wherein an angle θ1 between a first straight line and a second straight line in a plane perpendicular to a z direction satisfies a condition 90°<θ1≤180°, where the z direction is a direction in which the shaft extends, the first straight line is a straight line that connects a center of the first light source and a center of the shaft when seen from the z direction, and the second straight line is a straight line that connects a center of the second light source and the center of the shaft when seen from the z direction.

2. The batch culture device according to claim 1,
wherein the light sources include a light source group constituted by light sources that are disposed rotationally symmetrically around the shaft when the shaft and the light sources are seen along the shaft.

3. The batch culture device according to claim 1,
wherein the photosynthetic microorganism is a cyanobacterium.

4. The batch culture device according to claim 1,
wherein the reaction vessel has pressure resistance.

5. The batch culture device according to claim 1,
wherein the light sources include a light source that is disposed at a first position when the shaft and the light sources are seen along the shaft, and
wherein a distance between the side surface of the reaction vessel and the first position is shorter than a distance between the center of the shaft and the first position.

6. The batch culture device according to claim 1,
wherein the light sources include a light source that is disposed, when the inside of the reaction vessel is filled with a liquid whose volume is greater than or equal to 60% and less than or equal to 90% of a capacity of the reaction vessel, between a liquid surface formed by the liquid and a bottom surface of the reaction vessel.

7. The batch culture device according to claim 1,
wherein, when the inside of the reaction vessel is filled with a liquid whose volume is greater than or equal to 60% and less than or equal to 90% of a capacity of the reaction vessel, an area of light-emitting surfaces of the light sources that are in contact with the liquid is greater than or equal a half of a total area of the light-emitting surfaces.

8. The batch culture device according to claim 1,
wherein the light sources extend along the shaft.

9. The batch culture device according to claim 1, further comprising:
a bracket attached to the shaft,
wherein the light sources are suspended from the bracket.

10. The batch culture device according to claim 9,
wherein the bracket includes a clamp that is removable from the shaft and a bar that extends from the clamp toward the side surface of the reaction vessel when the clamp is fixed to the shaft.

11. The batch culture device according to claim 1, further comprising:
a temperature adjuster that is disposed along an inner surface of the reaction vessel and adjusts a temperature of the culture solution.

12. The batch culture device according to claim 11,
wherein the temperature adjuster adjusts an absolute value of a difference between an optimal growing temperature for the photosynthetic microorganism and the temperature of the culture solution to less than or equal to 5° C.

13. The batch culture device according to claim 11,
wherein the temperature adjuster is disposed so as surround an outer surface of the reaction vessel.

14. The batch culture device according to claim 11,
wherein the light sources extend along the shaft, and
wherein the temperature adjuster overlaps the light sources in a direction parallel to the shaft.

15. The batch culture device according to claim 1,
wherein the reaction vessel has an inner surface that is made of a metal or that is a mirror surface.

16. The batch culture device according to claim 1, further comprising:
an air passage that guides air to a lower portion of the reaction vessel and discharges air from an upper portion of the reaction vessel.

17. The batch culture device according to claim 1,
wherein the reaction vessel is capable of holding at least 0.4 m³ of the culture solution.

18. The batch culture device according to claim 1,
wherein the light sources emit light in a wavelength range of greater than or equal to 400 nm and less than or equal to 700 nm.

19. A batch culture device comprising:
a reaction vessel that is capable of holding a culture solution of a photosynthetic microorganism;
light sources that are disposed in an inside of the reaction vessel; and
a protruding member that protrudes from a side surface of the inside of the reaction vessel toward a center of the inside of the reaction vessel,
wherein the light sources are suspended from the protruding member,
wherein the light sources include a first light source and a second light source, and
wherein an angle θ1 between a first straight line and a second straight line in a plane perpendicular to a z direction satisfies a condition 90°<θ1≤180°, where the z direction is a direction parallel to the side surface of the inside of the reaction vessel, the first straight line is a straight line that connects a center of the first light source and a center of the inside of the reaction vessel when seen from the z direction, and the second straight line is a straight line that connects a center of the second light source and the center of the inside of the reaction vessel when seen from the z direction.
